(19) United States
(12) Patent Application Publication
(10) Pub. No.: US 2002/0107510 A1
(43) Pub. Date: Aug. 8, 2002

(54) Laser apparatus useful for myocardial revascularization

(76) Inventors: Robert R. Andrews, Norfolk, MA (US); Lawrence P. Brodsky, Tolland, CT (US); Richard P. Yeomans JR., Medway, MA (US); Italo J. Desantis, Medway, MA (US); Roger D. Dowd, Franklin, MA (US); Alan S. Kujawski, Webster, MA (US); Richard M. Morrell, Milford, MA (US); Charles Christopher Negus, Taunton, MA (US); Robert I. Rudko, Holliston, MA (US)

Correspondence Address: WILLIAM E. BOOTH, Fish & Richardson P.C., 225 Franklin Street, Boston, MA, 02110-2804, US

(21) Appl. No.: 09/777,915

(22) Filed: Feb. 5, 2001

(51) Int. Cl. A61B 18/18

(52) U.S. Cl. 606/10; 606/11; 372/9; 372/15; 372/29.013

(57) ABSTRACT

Cardiac laser surgery apparatus including a CO₂ slab laser, and a laser delivery system for delivering laser pulses from the laser to a patient's heart. Also isclosed are mirror adjustment devices for remotely adjusting mirrors sealed inside a laser housing; an RF power supply used to both preionize the laser gas, and to fire the laser by providing RF voltage pulses of longer duration than the preionizing RF voltage pulses; attaching laser optics to an integral fin along extruded laser housing; optical elements receiving a rectangular laser beam and converting it to a square laser beam of the desired dimensions; nonconductive spacers each connecting one electrode to the other in the laser chamber; barbed inserts in the electrodes that connect inductors to the electrodes; a plurality of resilient contact elements extending along a bottom of an electrode and supported on an internal ledge structure of the housing to provide good electrical connection between the electrode and the housing; isolation mounts between the housing and a frame; an air source including a compressor and an air filter, and a tube connected to the laser delivery system to deliver purge air from the source to the hand piece to clear smoke from the inside of the hand piece; and an upwardly extending lip around an output window at the bottom of the laser housing to limit collection of particles on the window.

### BACKGROUND OF THE INVENTION

The invention relates to laser apparatus useful for myocardial revascularization.

Lasers have been used in various medical applications. One such application is myocardial revascularization where laser pulses are directed to a patient's heart to provide minute channels to permit bloodflow and revascularization within the heart muscle. U.S. Pat. Nos. 5,109,388, 5,125,924, 5,125,926, 5,200,604, 5,219,347, 5,336,218, 5,509,822, 5,591,161, 5,617,258, 5,700,259, 5,724,975, 5,897,550, 6,113,587, and 6,132,422, which are hereby incorporated by reference, describe apparatus useful for myocardial revascularization.

### SUMMARY OF THE INVENTION

In one aspect, the invention features, in general, cardiac laser surgery apparatus including a CO₂ slab laser, and a laser delivery system for delivering laser pulses from the laser to a patient's heart.

Particular embodiments of the invention may include one or more of the following features. In particular embodiments, the laser delivery system includes a hand piece for delivering pulsed laser energy to the outside of a patient's heart to provide openings in the patient's heart for myocardial revascularization. The pulses are shorter than 100 ms and provide energy of between 8 and 80 Joules per pulse.

In another aspect, the invention features, in general, a laser assembly including an elongated housing defining a laser cavity having first and second ends and an opening at the first end, a mirror located inside of the housing at the first end, and a plate that is located outside of the housing. Adjustable connectors connect the plate to the first end at connector locations outside of the opening so as to adjust the angular orientation of the plate with respect to the housing. A support member has one end connected to the plate, another end carrying the mirror, and an intermediate portion passing through the opening. A metal bellows structure surrounds the support member and has one end that is sealed to the first end of the housing around the opening and another end that is sealed to the plate around the intermediate portion and inside of the connector locations so as to provide a vacuum-tight seal between the housing and the plate around the opening. With this arrangement, the angular orientation of the mirror with respect to the housing can be adjusted by adjusting the adjustable connectors to adjust the orientation of the plate.

Particular embodiments of the invention may include one or more of the following features. In particular embodiments there are two adjustable connectors and a third connector connecting the plate to the end of the housing. The adjustable connectors have screw threads to adjust the distance between the plate and the end of the housing at the respective connector locations. The assembly includes slab electrodes within the housing, the electrodes being spaced from each other by a gap that is aligned with the mirror. There is a second mirror at the other end of the housing, the second mirror being aligned with the gap. A bracket that is secured to the end of the housing supports the electrodes.

In another aspect, the invention features, in general, a laser assembly comprising an elongated housing defining a laser cavity having first and second ends, and openings at the first end, a mirror located inside of the housing at the first end, and adjustment devices that are sealably connected at the openings, the adjustment devices adjustably positioning the mirror with respect to the first end so as to adjust the angular orientation of the mirror with respect to the housing, the adjustment devices having engagement portions accessible outside of the first end.

Particular embodiments of the invention may include one or more of the following features. In particular embodiments, each adjustment device includes a rotary motion vacuum feedthrough device sealably connected in a respective opening and a screw thread device that is connected to be rotated by the rotary motion feedthrough device so as to adjust the position of the mirror with rotation of the screw thread device. The adjustment device adjusts the orientation with respect to a first axis, and another the adjustment device adjusts the orientation with respect to a second axis that is perpendicular to the first axis. The assembly includes slab electrodes within the housing, the electrodes being spaced from each other by a gap that is aligned with the mirror.

In another aspect, the invention features, in general, laser apparatus including a laser housing having laser electrodes, mirrors and a laser gas therein, an RF power supply connected to supply RF voltage pulses to the electrodes, and a control circuit that controls the RF power supply to preionize the laser gas by supplying RF voltage pulses provided by the RF power supply, and to fire the laser by providing RF voltage pulses of longer duration than the RF voltage pulses.

Particular embodiments of the invention may include one or more of the following features. In particular embodiments, the RF power supply includes an amplifier that provides output pulses, an RF source, and a switch that connects the RF source to an input of the amplifier. The laser apparatus delivers 800 watts. The laser gas is a mixture of carbon dioxide, helium, nitrogen and xenon.

In another aspect, the invention features, in general, laser apparatus including a housing made of a metal extrusion having a uniform cross-section and defining an elongated laser chamber therein extending along a longitudinal axis. The housing has a fin extending outward from one side of the housing and linearly along the housing parallel to the longitudinal axis. Laser focusing optics are mounted on the fin along an optical axis that is parallel to the longitudinal axis, and laser directing elements direct a laser beam generated in the chamber to the focusing optics.

In another aspect, the invention features, in general, laser apparatus including a slab laser beam generator generating a rectangular laser beam having a width dimension and a thickness dimension, and a plurality of optical elements receiving the rectangular laser beam and converting it to a square laser beam of the desired dimensions.

Particular embodiments of the invention may include one or more of the following features. In particular embodiments, the rectangular laser beam is 2 mm by 10 mm, and wherein the square laser beam is 9.5 mm by 9.5 mm. The optical elements include a first cylindrical lens to expand the beam in the thickness dimension to an expanded beam and a second cylindrical lens to collimate the expanded beam from the first cylindrical lens. The optics include a spatial filter including a third focusing lens and a fourth focusing lens and a slit between the third focusing lens and the fourth focusing lens. There is a shutter between the third focusing lens and set fourth focusing lens. The first cylindrical lens is a negative lens, and the second cylindrical lens is a positive lens.

In another aspect, the invention features, in general, a slab laser including a pair of elongated spaced apart electrodes having opposed planar surfaces defining a discharge region between the opposed planar surfaces, the electrodes having aligned side surfaces extending along a longitudinal axis thereof, and a plurality of nonconductive spacers each connecting one electrode to the other electrode at the side surfaces and being spaced from adjacent spacers along the side surfaces. The spacers have relieved portions facing the discharge region between first and second planes passing through the planar surfaces, the relieved portions including at least one projecting portion extending toward the discharge region and having an apex closer to the discharge region than adjacent portions of the relieved portions.

Particular embodiments of the invention may include one or more of the following features. In particular embodiments, the apex is on the side of a third plane through the sides of the electrodes outside of the discharge region and spaced from the third plane. The spacer can have more than one projecting portion extending toward the discharge region and having an apex region. The apex converges to a line.

In another aspect, the invention features, in general, a slab laser including a pair of elongated spaced apart electrodes having opposed planar surfaces defining a discharge region between the opposed planar surfaces, the electrodes having aligned side surfaces extending along a longitudinal axis thereof and a plurality of nonconductive spacers each connecting one the electrode to the other the electrode at the side surfaces and being spaced from adjacent spacers along the side surfaces. A plurality of barbed inserts are located in the side surfaces and spaced from adjacent barbed inserts along the inside surfaces. A plurality of inductors each have one end inserted into a barbed insert on one electrode and another end inserted into a barbed insert on the other electrode.

In another aspect, the invention features, in general, a slab laser including an elongated housing having an internal ledge structure along at least one side, a pair of elongated spaced apart electrodes having opposed planar surfaces defining a discharge region between the opposed planar surfaces, and a plurality of resilient contact elements extending along a bottom of a the electrode and supported on the internal ledge structure to provide good electrical connection between the electrode and the housing.

In another aspect, the invention features, in general, laser apparatus comprising a housing defining an elongated laser chamber therein extending along a longitudinal axis, a frame having support portions supporting the housing, and isolation mounts between the support portions and the housing.

Particular embodiments of the invention may include one or more of the following features. In particular embodiments, wheels are attached to the frame. The bumpers include a vertically oriented bumper and a horizontally oriented bumper, preferably two vertically oriented bumpers and two horizontally oriented bumpers.

In another aspect, the invention features, in general, myocardial revascularization apparatus including a laser, a laser delivery system for delivering laser pulses from a hand piece to a patient's heart, an air source including a compressor and an air filter, and a tube connected to the laser delivery system to deliver purge air from the source to the hand piece to minimize debris on the lens cell focusing optic inside the hand piece.

In another aspect, the invention features, in general, laser apparatus including a housing defining an elongated laser chamber aligned with a vertical axis. The housing has a laser output window at the lower end of the housing, and an upwardly extending lip around the window to limit collection of particles on the window.

Embodiments of the invention may include one or more of the following features. The use of a CO₂ slab laser for cardiac surgery apparatus permits one to produce high energy from a small package that is sealed off and quiet, and is of low cost. There is no need to provide a supply of continuous gas flow as in some other types of CO₂ lasers. Isolation mounts isolate the entire optic train from the rest of the system, improving optical alignment, stability, and reliability. The use of an air compressor and a filter avoids the need to provide a tank of compressed gas, e.g., as required when carbon dioxide is used as the purge gas. The same power supply and the same voltage are used to both preionize and to the fire laser. The projections on the surface of the ceramic spacer exposed to the discharge region avoid electrical tracking along the spacer.

Other features and advantages of the invention will be apparent from the following description of a particular embodiment thereof and from the claims.

### BRIEF DESCRIPTION OF THE DRAWINGS

FIG. 1 is a side view showing laser apparatus useful for myocardial revascularization.

FIG. 2 is a perspective view of a frame and laser chamber and mount components of the FIG. 1 apparatus.

FIG. 3 is a perspective view of a laser chamber device of the FIG. 1 apparatus.

FIG. 4 is an exploded perspective view of components of the FIG. 3 laser chamber device.

FIG. 4A is an enlarged perspective view of a portion of a contact strip of the FIG. 3 laser chamber device.

FIG. 5 is an elevation of the end of a housing and adjustment components at the end of the FIG. 3 laser chamber device.

FIG. 6 is a vertical sectional view, taken at 6-6 of FIG. 5, of the FIG. 5 housing end and adjustment components.

FIG. 7 is a vertical sectional view, taken at 7-7 of FIG. 3, of the FIG. 3 laser chamber device.

FIG. 7A is a side sectional view of a lower plate with an output window of the FIG. 3 device.

FIG. 8 is an exploded partial perspective view of an end of the FIG. 3 laser chamber device.

FIG. 9 is a partial vertical sectional view, taken at 9-9 of FIG. 4, of the electrode components of the FIG. 3 laser chamber device.

FIG. 10 is an elevation of a ceramic electrode spacer used in the FIG. 3 laser chamber device.

FIG. 11 is a side view of the FIG. 10 spacer.

FIG. 12 is a top view of the FIG. 10 spacer.

FIG. 13 is a perspective view of the FIG. 10 spacer.

FIG. 14 is an elevation of an alternative ceramic electrode spacer used in the FIG. 3 laser chamber device.

FIG. 15 is a side view of the FIG. 14 spacer.

FIG. 16 is a top view of the FIG. 14 spacer.

FIG. 17 is a perspective view of the FIG. 14 spacer.

FIG. 18 is a perspective view of an alternative mirror positioning arrangement.

FIG. 19 is a block diagram showing electrical circuitry for powering the FIG. 1 laser apparatus.

FIG. 20 is a diagram showing optics used in the FIG. 1 apparatus.

FIG. 21 is a diagram showing a purge gas supply system used in the FIG. 1 apparatus.

FIG. 22 is a timing diagram for preionizing and firing the FIG. 1 laser apparatus.

### DESCRIPTION OF A PARTICULAR EMBODIMENT

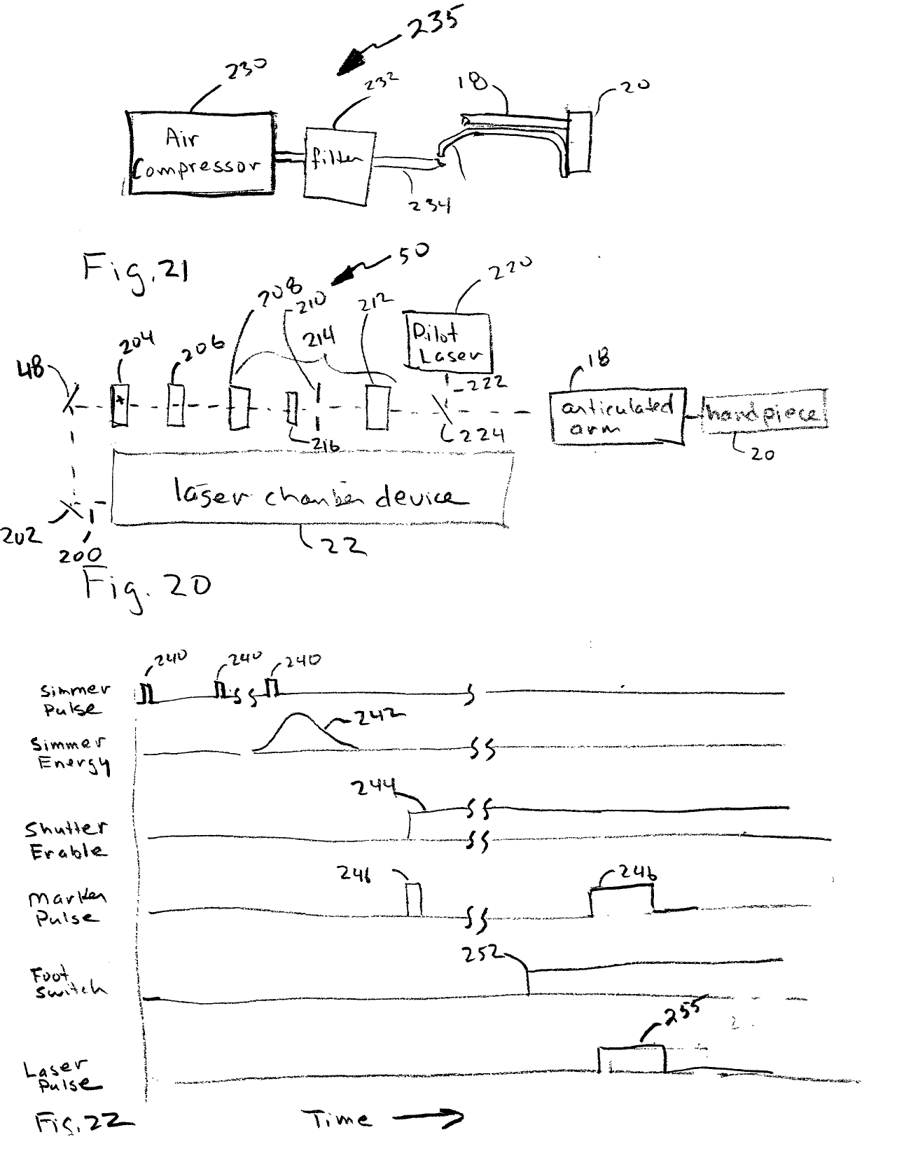

Referring to FIG. 1, there is shown laser apparatus 10 for performing myocardial revascularization. Apparatus 10 is portable on wheels 12 and includes an internal computer (166 on FIG. 19) that controls the laser and is user controlled via display/touch screen 16. Apparatus 10 also includes articulated arm 18 that carries hand piece 20 (not shown in FIG. 1; see FIG. 20) at the end of arm 18 for delivering laser pulses to the outside of a patient's heart, e.g., as described in the above identified patents.

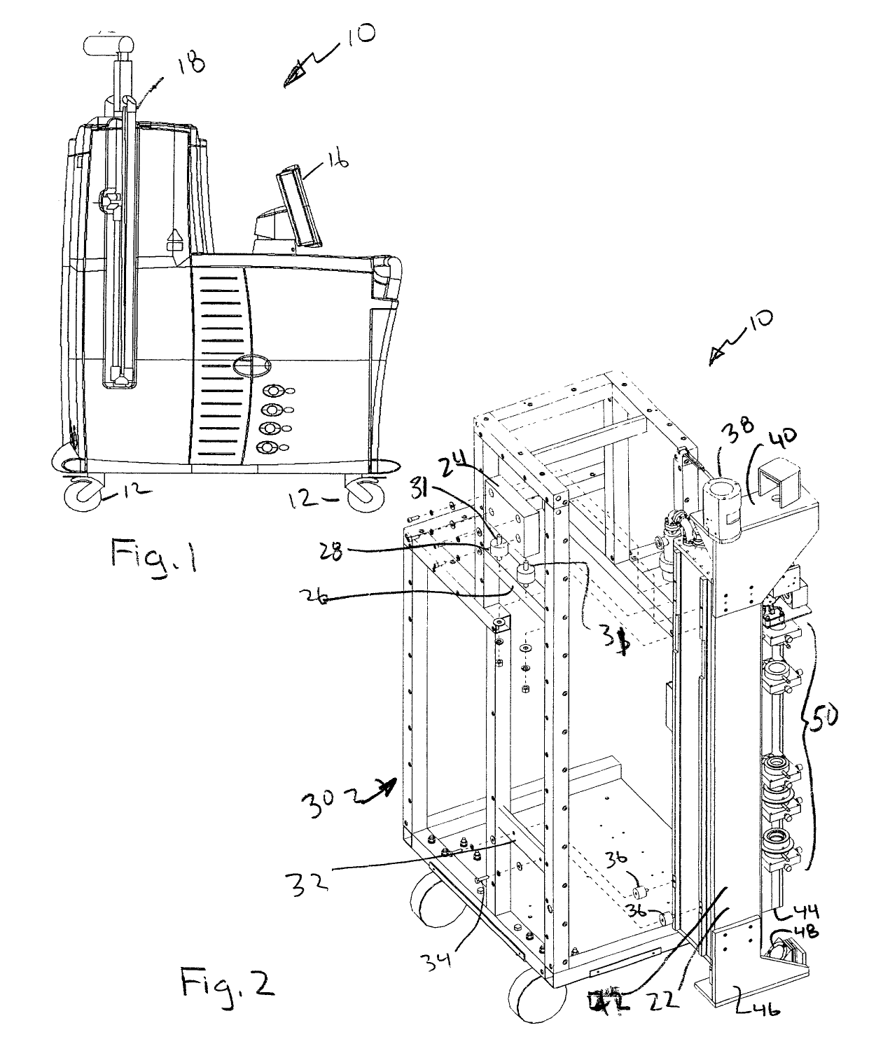

Referring to FIG. 2, apparatus 10 includes laser chamber 22, which is secured to upper mounting plate 24, which is in turn supported on crossbar 26 of frame 30 via bolts 31 which pass through isolation mounts 28 to provide shock absorption for laser chamber 22 on frame 30. The bottom part of laser chamber 22 is secured to lower crossbar 32 via bolts 34, which pass through lower isolation mounts 36. Isolation mounts 28 and 36 are made of elastomeric material. Articulated arm 18 is secured to base 38 on the top of support bracket 40 on the top of laser chamber 22. Laser chamber 22, the laser resonator of apparatus 10, is vertically mounted to minimize the amount of floor space taken up by apparatus 10. Isolation mounts 28 isolate the entire optic train (from laser resonator to the articulated arm) from the rest of the system. In so doing, optical alignment, stability, and reliability are maximized.

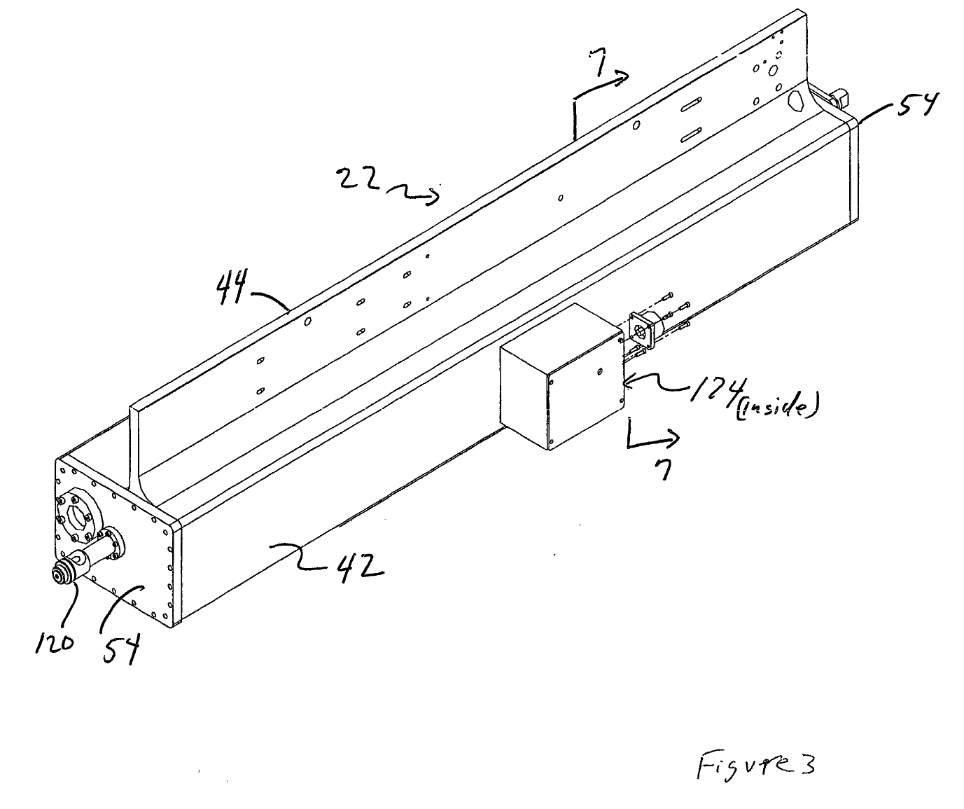

Referring to FIGS. 2 and 3, laser chamber 22 includes an elongated extrusion 42 having an integral fin 44 on which optical elements 50 are mounted. Lower bracket 46 is secured to the bottom of extrusion 42 and carries two mirrors, only one of which, mirror 48, is shown in FIG. 2. These mirrors direct the laser beam exiting vertically downward from the bottom of laser chamber 22 first sideways and then upward through optical elements 50. Laser chamber 22 includes an end plate 52 that closes off one end of extrusion 42 and an end plate 54 that closes off the other end of extrusion 42.

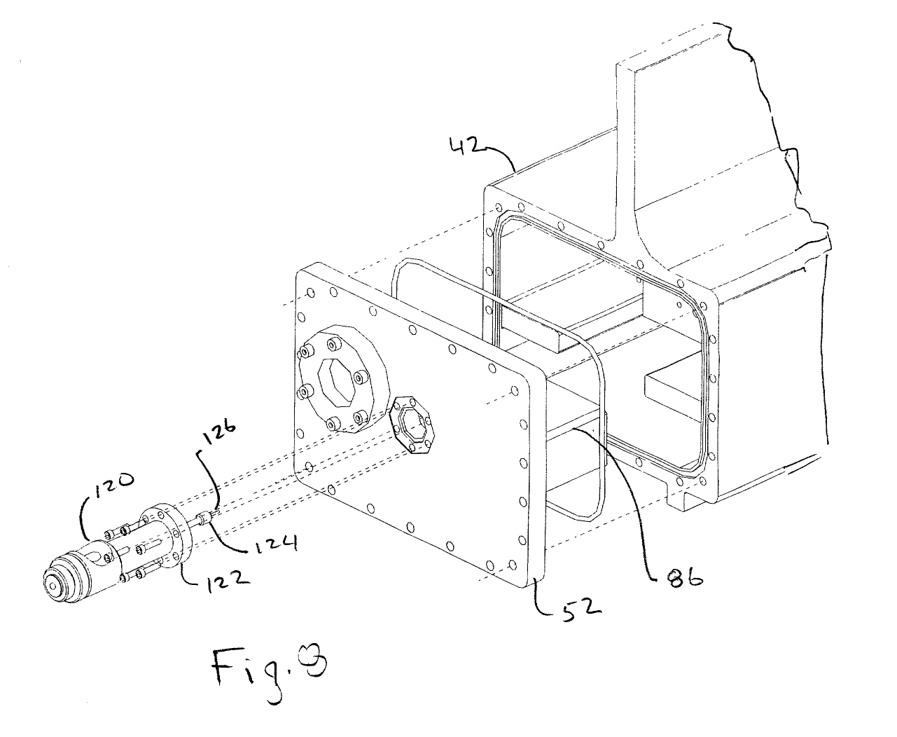

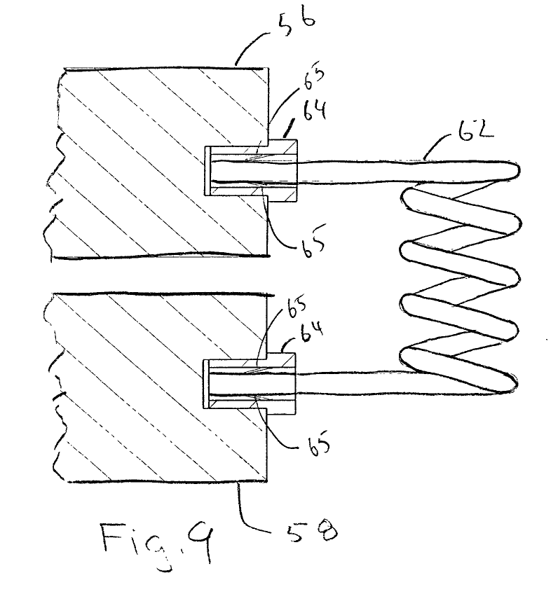

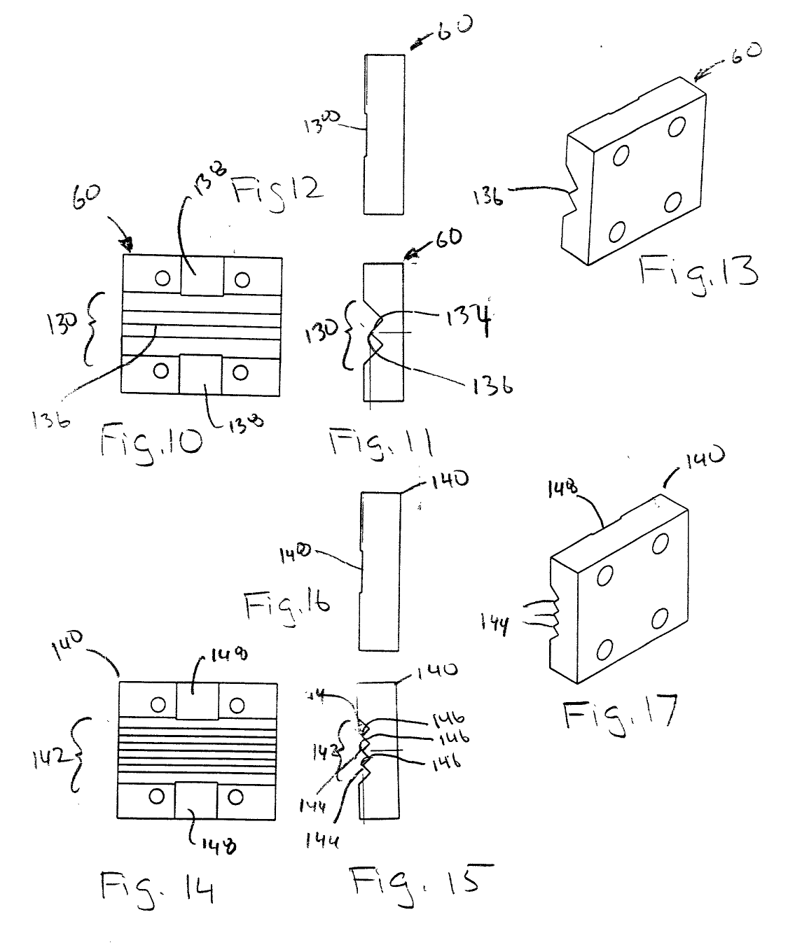

Referring to FIG. 4, the internal components of laser chamber 22 are shown with end plate 54. The internal components include two slab electrodes 56, 58 that are secured to and spaced from each other by ceramic electrode spacers 60 (shown in detail in FIGS. 10-17) that are secured to electrodes 56, 58 along side surfaces 61 along the lengths of electrodes 56, 58. Electrodes 56, 58 are 10 cm wide, 90 cm long and are spaced by 2.0 mm, providing a 90 cm by 10 cm by 2.0 mm discharge region 132 (FIG. 7) between them and a 2 mm by 10 cm rectangular laser beam, though only a 9.5 mm portion is sampled and provided as a 2 mm by 10 mm output beam. Electrodes 56, 58 are electrically connected to each other via inductors 62 that are spaced along side surfaces 61 of electrodes 56, 58. Electrode 58 is fixedly secured at one end to bracket 66, which is fixedly secured to housing end plate 54. The other end of electrode 58 is fixedly secured to support member 68, which has extension 70 carrying resilient top contact 72 and resilient side contact 74. Extension 70 fits within a receptacle secured to end plate 52 (shown in FIG. 3) and provides support and electrical connection for this end of electrode 58 and also permits deflection in the X and Y axes to accommodate thermal expansion/retraction of electrodes 56, 58 within laser chamber 22. Resilient contact strips, one of which, contact strip 80, is shown in FIG. 4, are connected to the bottom of electrode 58 along the two side surfaces 61 to provide electrical contact to ledges 110, 112 of extrusion 42 (see FIG. 7). FIG. 4A shows an enlarged view of a contact strip 80 oriented upside down. Strip 80 includes a flat base 81 made of brass with a gold finish, and a plurality of resilient contacts 83 made of beryllium copper with a gold finish. Contacts 83 are bent and have hooked ends that extend through holes in flat base 81.

Still referring to FIG. 4, the internal components of laser chamber 22 also include mirrors 82, 84. When assembled within laser chamber 22, mirrors 82, 84 are aligned with the gap between electrodes 56, 58. Mirror 84 is secured to adjustment device 86 which is in turn carried on a bracket 88 secured to end plate 52. Mirror 82 is carried on a support member 90, which is connected to external plate 92.

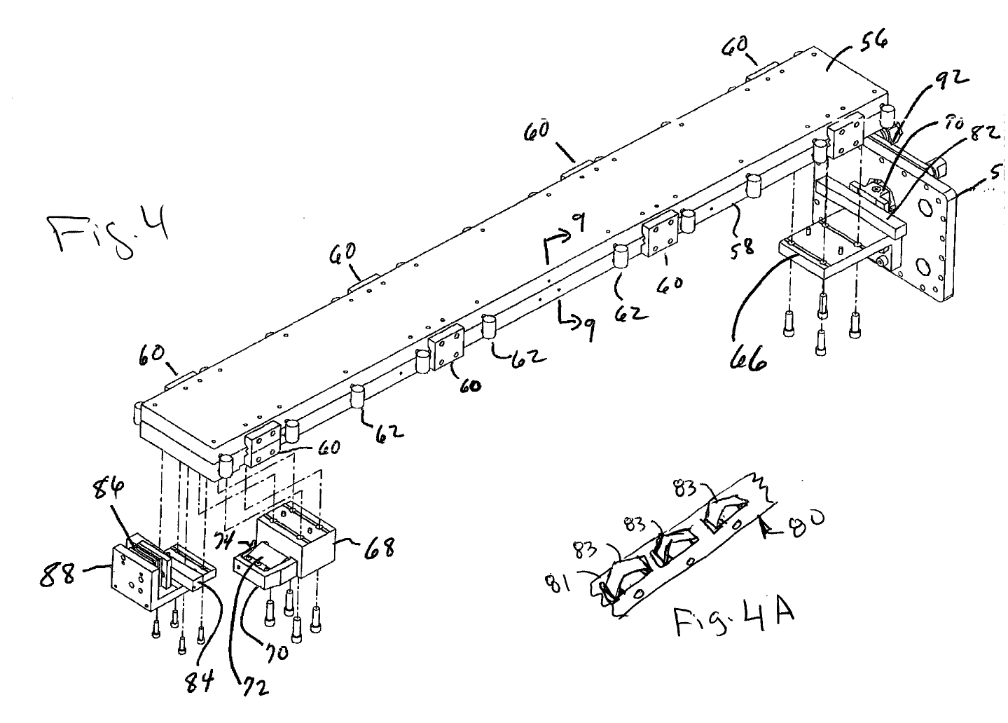

Referring to FIGS. 5 and 6, mirror support 90 is fixedly connected to external plate 92 via support member 94. Support member 94 passes through opening 96 in end plate 54. External plate 92 is secured to end plate 54 by a nonadjustable connector 98 and two adjustable connectors 100, 102. Adjustable connector 102 includes a threaded shaft 106 that adjusts the distance between external plate 92 and end plate 54 at connector 102. Adjustable connector 100 similarly includes a threaded shaft (not shown on FIG. 6) that adjusts the distance between external plate 92 and end plate 54 at connector 100. Rotation of adjustable connector 100 adjusts the angular orientation of mirror 82 with respect to the Y-axis, while rotation of adjustable connector 102 adjusts mirror 82 with respect to the X-axis. The Y-axis corresponds to an unstable resonator and the X-axis corresponds to the slab laser resonator.

FIG. 7 shows the support of electrode 58 on ledges 110, 112 via contact strips 80. Contacts 83 (not visible in FIG. 7) are depressed by the electrodes. Referring to FIG. 7A, lower plate 52 has an upwardly extending lip 53 around output window 55 to limit collection of particles on window 55.

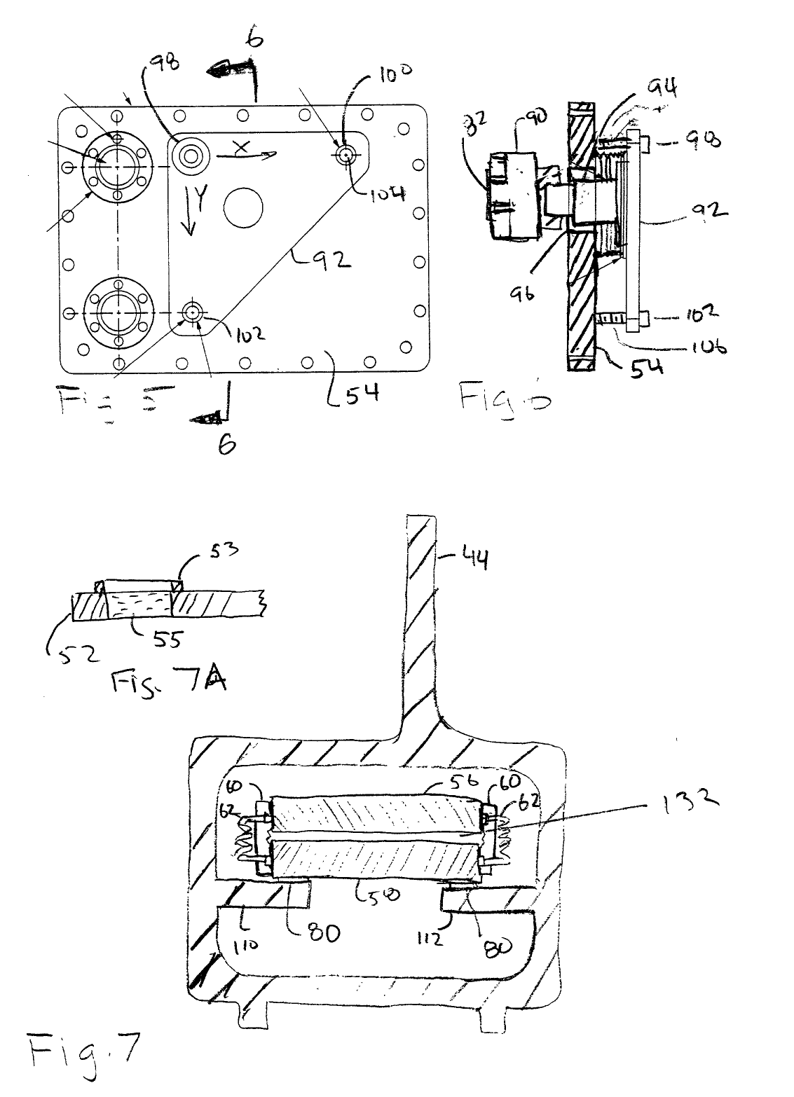

FIG. 8 shows rotary motion feedthrough device 120, which is secured to end plate 52 to provide adjustment of mirror 84 with respect to the X-axis. Base 122 of device 120 is sealably secured to end plate 52. Internal member 124 of device 20 has an Allen head wrench on 26 at the end. This connects with a threaded adjustment device (not shown) that moves support 86 and mirror 84 to adjust the angular orientation with respect to the X-axis for fine tuning the laser output mode.

FIG. 9 shows an inductor 62 secured to electrodes 56, 58 via barbed inserts 64. Inserts 64 are press-fit into electrodes 56, 58 and have barbs 65 that engage the ends of inductor 62, preventing removal and digging into the inductor to provide good electrical contact.

FIGS. 10-13 show an embodiment of ceramic spacer 60. It includes a relieved portion 130 that faces the discharge region 132 (FIG. 7) between electrodes 56, 58. Relieved portion 130 includes projection 134 that extends toward discharge region 132 and has apex 136 closer to discharge region 132 than adjacent portions of relieved portion 130. Spacer 60 also has a vertically extending relieved portion 138. Apex 136 is further away from discharge region 132 than a plane passing through side surfaces 61.

FIGS. 14-17 show alternative ceramic spacer 140. It includes a relieved portion 142 that faces the discharge region 132 and has three projections 144 that extend toward the discharge region 132 and have apexes 146 closer to discharge region 132 than adjacent portions of relieved portion 142. It also has a vertically extending relieved portion 148. Apexes 146 are further away from discharge region 132 than a plane passing through side surfaces 61.

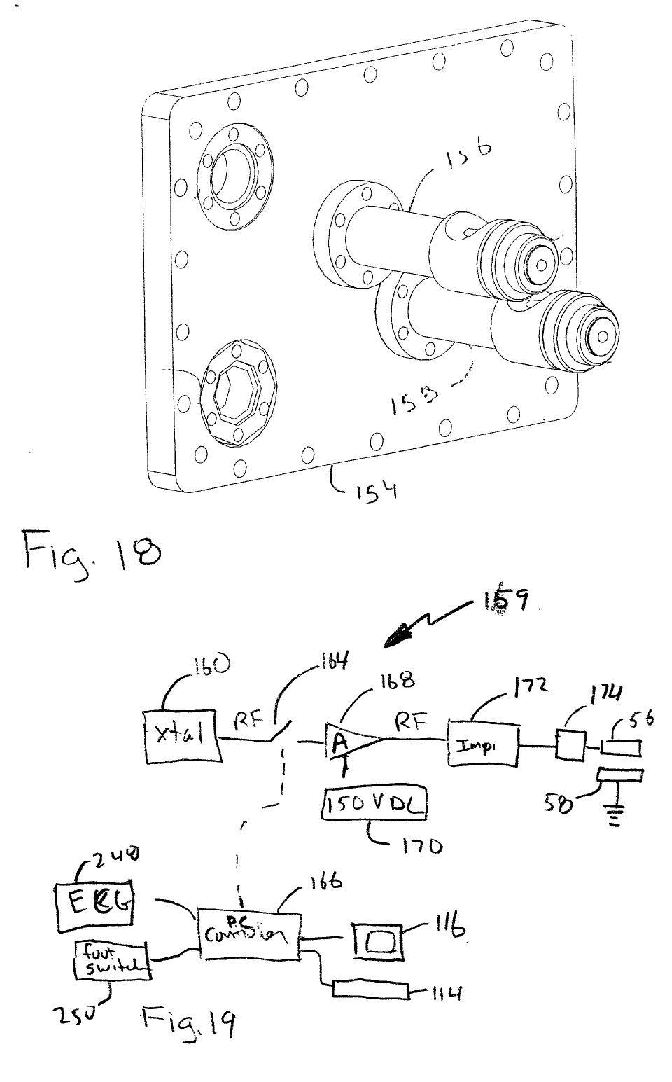

FIG. 18 shows an alternative mechanism for providing angular orientation of mirror 82 with respect to end plate 154, which replaces end plate 54 in this alternative. Instead of external plate 92 and adjustable connectors 100, 102, the alternative mechanism employs two rotary feadthrough devices 156, 158, the former being used to adjust in the x-axis, the latter being used to adjust in the y-axis. As with feedthrough mechanism 120, mechanisms 156, 158 each have an Allen wrench head used to rotate respective threaded adjustment devices (not shown) that move a support for mirror 82 with respect to the x-axis and y-axis.

FIG. 19 shows circuitry 159 used to provide power to electrodes 56, 58 of laser chamber 22. Circuitry 159 includes 81.36 MHz crystal 160, which provides an RF output to switch 164, controlled by controller 166 (a computer), which is connected to touch screen/display 116 and keyboard 114. Switch 164 is connected to the input of amplifier 168, which is powered by a 150 VDC power source 170. The RF output of amplifier 168 passes through impedance matching circuit 172 and feedthrough 174 (through the sealed opening in extrusion 45; see FIG. 3) to electrode 56. Impedance matching circuit 172 includes low inductance capacitors and an RF connector enclosed within an RF shielded box. Circuit 172 closely matches the impedance of the laser resonator to that of the RF power supply to maximize power transfer from the supply to load. Electrode 58 is connected via contact strips 80 to ground. Controller 166 provides on/off control signals to switch 164 to provide both low duty cycle simmer pulses (200 microseconds, 16 W average power, at 50 Hz repetition), used to preionize the carbon dioxide gas in laser chamber 22, and to provide the longer pulses of desired length for desired energy (from 10 ms to 99 ms to provide from 8 to 80 Joules, respectively) to fire laser chamber 22 to provide a laser beam to create channels in a patient's heart tissue. Thus the same power supply and the same voltage are used to both preionize and to fire laser chamber 22.

FIG. 20 illustrates optical elements 50 used to convert the 2 mm by 9.5 mm rectangular laser beam output by laser chamber 22 into a 9.5 mm by 9.5 mm square beam that is launched into articulated arm 18 and delivered to hand piece 20. The rectangular beam 200 exiting from laser chamber 22 is reflected by mirrors 202, 48 to cylindrical lenses 204, 206, which are beam correcting optics that take the rectangular beam with different convergence in each axis and convert it to a square beam with the same divergence in each axis. A cylindrical lens can focus the beam on one axis without affecting the other axis. Because the unstable beam (X-axis) exits the laser with the required dimension, it is only necessary to correct the beam in the Y-axis. If it were necessary to resize the beam in both directions, additional cylindrical lenses would be used. Cylindrical lens 204 is a negative lens with a 35.1 cm focal length. This lens causes the slab laser beam to expand. Cylindrical lens 206 is a positive lens with a focal length of 35.5 cm. This lens takes the expanding beam and makes it approximately the same size in both the X and Y directions.

After beam 200 has been made square and collimated, it needs to be spatially filtered to remove higher order modes. It then needs to be adjusted to the correct size and convergence angle so it can be launched into articulated arm 18 and have the desired characteristics when delivered at hand piece 20. Lenses 208, slit 210, and end lens 212 act as a spatial filter 214. Beam 200 is focused into slit 210 using lens 208, which has a 20.3 cm focal length. After slit 210, lens 212, which also has a 20.3 cm focal length, is used to launch the beam into articulated arm 18 slightly converging. Shutter/detector 216 is located between lens 208 and shutter 210 and is used to shutter (i.e., to block or transmit) beam 200 and also includes a laser energy detector to detect when there is transmission of laser energy as a result of preionization simmer pulses. Pilot laser 220 is a low-power laser diode (5 mW maximum output at a wavelength of 635 nm) that provides a positioning laser beam 222 to beam combiner 224, which directs the positioning laser beam into articulated arm 18.

FIG. 21 illustrates a purge gas supply system used to provide purge gas to hand piece 20 to minimize debris on lens cell focusing optic and clear smoke from hand piece 20 during use. The system 235 includes air compressor 230, filter 232, and tubing 234, which provides filtered air through tubing 234 to the end of hand piece 20. Filter 232 includes a 0.3-micron pore size filter. The use of an air compressor and a filter avoids the need to provide a tank of compressed gas, e.g., as required when carbon dioxide is used as the purge gas.

FIG. 22 shows the control pulses and sensed energy used in firing laser apparatus 10. The laser needs to be warmed up whenever it is first turned on and whenever it is in an armed state. If the laser head is cold, a typical warm-up time is approximately 15 to 30 seconds. When the laser has been firing, the warm-up is as fast as one or two seconds. The warm-up involves providing low duty cycle pulses in order to preionize the carbon dioxide gas. Simmer pulses 240 are provided at 50 Hz to switch 164 by controller 166 (FIG. 19). This permits the RF signal from crystal 160 to be amplified at amplifier 168 and applied to electrode 56. The simmer pulses fire until the laser gas is ionized and produces laser energy. After the carbon dioxide gas has preionized, the laser chamber device will provide an output, and simmer energy 242 will be detected at shutter/detector 216. (FIG. 20) When the presence of laser energy is detected, the simmer pulses are turned off and the laser is ready to fire. Computer 166 provides a shutter enable signal 244 that permits shutter/detector 216 to be opened when the operator desires to fire laser apparatus 10. Marker pulses 246 are provided by a marker circuitry within PC controller 166, providing pulses after detecting each R wave in the patient's ECG signal from ECG 248. When the operator depresses foot switch 250, foot switch signal 252 is generated. Upon the occurrence of the next marker pulse 246, a laser firing pulse 254 is generated, causing laser chamber 22 to output a laser beam. In laser chamber 22, the RF energy is input to electrode 56, the floating electrode, to generate an electrical discharge between electrodes 56, 58. This discharge creates photonic energy from the carbon dioxide molecules, which resonates between mirrors 82, 84. This energy is leaked out past one side of mirror 84 and exits through a window in end plate 52. The beam 202 is then focused and shaped at optics 50 and launched into articulated arm 18 for delivery to hand piece 20 (FIG. 20) and the patient's heart. The apparatus produces up to 800 watts of power from the end of articulated arm 18. The same power is delivered each pulse. By increasing or decreasing the pulse width (the actual time the laser is on) the desired energy can be obtained. The laser is capable of producing pulses of energy ranging from 8 to 80 joules in pulse durations of 10 to 99 milliseconds.

The use of a CO₂ slab laser for cardiac surgery apparatus permits one to produce high energy from a small package that is sealed off and quiet, and is of low cost. There is no need to provide a supply of continuous gas flow as in some other types of CO₂ lasers.

The undulations on the surface of the ceramic spacer exposed to the discharge region avoid electrical tracking along the spacer.

The isolation mounts provide for vibration isolation vertically and horizontally.

Other embodiments of the invention are within the scope of the appended claims.

What is claimed is:

1. Cardiac laser surgery apparatus comprising
a CO₂ slab laser, and
a laser delivery system for delivering laser pulses from said laser to a patient's heart.

2. The apparatus of claim 1 wherein said laser delivery system includes a hand piece for delivering pulses to the outside of a patient's heart to provide openings in the patient's heart for myocardial revascularization.

3. The apparatus of claim 1 wherein said pulses are shorter than 100 ms and provide energy of between 8 and 80 Joules per pulse.

4. The apparatus of claim 1 wherein said laser delivery system is synchronized to the heart beat to fire when the heart is electrically insensitive to reduce the chanceofarrhythmia.

5. The apparatus of claim 4 wherein said laser starts firing on the R wave and stops before the T wave.

6. A method of cardiac laser surgery comprising
operating a CO₂ slab laser to output laser pulses, and
delivering said laser pulses to a patient's heart.

7. The method of claim 6 wherein said delivering provides openings in the patient's heart for myocardial revascularization.

8. The method of claim 6 wherein said pulses are shorter than 100 ms and provide energy of between 8 and 80 Joules per pulse.

9. The method of claim 6 wherein said laser delivery system is synchronized to the heart beat to fire when the heart is electrically insensitive to reduce the chanceofarrhythmia.

10. The method of claim 9 wherein said laser starts firing on the R wave and stops before the T wave.

11. A laser assembly comprising
an elongated housing defining a laser cavity having first and second ends, and an opening at said first end,
a mirror located inside of said housing at said first end,
a plate that is located outside of said housing,
adjustable connectors adjustably connecting said plate to said first end at connector locations outside of said opening so as to adjust the angular orientation of said plate with respect to said housing,
a support member having one end connected to said plate, another end carrying said mirror, and an intermediate portion passing through said opening, and
a metal bellows structure surrounding said support member and having one end that is sealed to said first end of said housing around said opening and another end that is sealed to said plate around said intermediate portion and inside of said connector locations so as to provide a vacuum-tight seal between said housing and said plate around said opening,
whereby the angular orientation of said mirror with respect to said housing can be adjusted by adjusting said adjustable connectors to adjust the orientation of said plate.

12. The assembly of claim 11 wherein there are two said adjustable connectors and a third connector connecting said plate to said end of said housing.

13. The assembly of claim 11 wherein said adjustable connectors have screw threads to adjust the distance between said plate and said end of said housing at said respective connector locations.

14. The assembly of claim 11 further comprising slab electrodes within said housing, said electrodes being spaced from each other by a gap that is aligned with said mirror.

15. The assembly of claim 14 further comprising a second mirror at the other end of said housing, said second mirror being aligned with said gap.

16. The assembly of claim 14 further comprising a bracket that is secured to said end of said housing and supports said electrodes.

17. Myocardial revascularization apparatus comprising the laser assembly of claim 11 and a laser delivery system for delivering laser pulses to a patient's heart to provide openings in the patient's heart.

18. The apparatus of claim 17 wherein said laser delivery system includes a hand piece for delivering pulses to the outside of a patient's heart.

19. A laser assembly comprising
an elongated housing defining a laser cavity having first and second ends, and openings at said first end,
a mirror located inside of said housing at said first end, and
adjustment devices that are sealably connected at said openings, said adjustment devices adjustably positioning said mirror with respect to said first end so as to adjust the angular orientation of said mirror with respect to said housing, said adjustment devices having engagement portions accessible outside of said first end.

20. The assembly of claim 19 wherein each said adjustment device includes a rotary motion feedthrough device sealably connected in a respective said opening and a screw thread device that is connected to be rotated by said rotary motion feedthrough device so as to adjust the position of said mirror with rotation of said screw thread device.

21. The assembly of claim 19 wherein one said adjustment device adjusts the orientation with respect to a first axis, and another said adjustment device adjusts the orientation with respect to a second axis that is perpendicular to said first axis.

22. The assembly of claim 19 further comprising slab electrodes within said housing, said electrodes being spaced from each other by a gap that is aligned with said mirror.

23. The assembly of claim 22 further comprising a second mirror at the other end of said housing, said second mirror being aligned with said gap.

24. The assembly of claim 22 further comprising a bracket that is secured to said housing and supports said electrodes.

25. Myocardial revascularization apparatus comprising the laser assembly of claim 19 and a laser delivery system for delivering laser pulses to a patient's heart to provide openings in the patient's heart.

26. The apparatus of claim 25 wherein said laser delivery system includes a hand piece for delivering pulses to the outside of a patient's heart.

27. A method of operating a laser comprising
providing a laser and an RF power supply for said laser,
preionizing a laser using RF voltage pulses provided by an RF power supply, and
after said laser has been preionized, firing said laser using said RF power supply to provide an output laser pulse of longer duration than said RF voltage pulses.

28. The method of claim 27 wherein said RF voltage pulses have a low duty cycle during said preionizing step.

29. The method of claim 27, further comprising delivering said laser pulses to a patient's heart.

30. Laser apparatus comprising
a laser housing having laser electrodes, mirrors and a laser gas therein,
an RF power supply connected to supply RF voltage pulses to said electrodes, and
a control circuit that controls said RF power supply to preionize said laser gas by supplying RF voltage pulses provided by said RF power supply, and to fire said laser by providing RF voltage pulses of longer duration than said RF voltage pulses.

31. The laser apparatus of claim 30 wherein said RF power supply includes an amplifier that provides output pulses, an RF source, and a switch that connects said RF source to an input of said amplifier.

32. The system of claim 30 wherein said laser apparatus delivers 800 watts.

33. The system of claim 30 wherein said laser gas is a mixture of carbon dioxide, helium, nitrogen, and xenon.

34. Myocardial revascularization apparatus comprising the laser apparatus of claim 30 and a laser delivery system for delivering laser pulses to a patient's heart to provide openings in the patient's heart.

35. The apparatus of claim 34 wherein said laser delivery system includes a hand piece for delivering pulses to the outside of a patient's heart.

36. Laser apparatus comprising
a housing made of a metal extrusion having a uniform cross-section and defining an elongated laser chamber therein extending along a longitudinal axis,
said housing having a structure extending outward from one side of said housing and linearly along said housing parallel to said longitudinal axis,
laser focusing optics mounted on said structure along an optical axis that is parallel to said longitudinal axis, and
laser directing elements directing a laser beam generated in said chamber to said focusing optics.

37. Myocardial revascularization apparatus comprising the laser apparatus of claim 36 and a laser delivery system for delivering laser pulses to a patient's heart to provide openings in the patient's heart.

38. The apparatus of claim 37 wherein said laser delivery system includes a hand piece for delivering pulses to the outside of a patient's heart.

39. Laser apparatus comprising
a slab laser beam generator generating a rectangular laser beam having a width dimension and a thickness dimension, and
a plurality of optical elements receiving said rectangular laser beam and converting it to a square laser beam of the desired dimensions.

40. The laser apparatus of claim 39 wherein said rectangular laser beam is 2 mm by 10 mm, and wherein said square laser beam is 9.5 mm by 9.5 mm.

41. The laser apparatus of claim 39 wherein said optical elements include a first cylindrical lens to expand said beam in said thickness dimension to an expanded beam and a second cylindrical lens to collimate the expanded beam from the first cylindrical lens.

42. The laser apparatus of claim 41 wherein said optical elements further include a spatial filter.

43. The laser apparatus of claim 42 wherein said spatial filter includes a third focusing lens and a fourth focusing lens and a slit between said third focusing lens and said fourth focusing lens.

44. The laser apparatus of claim 43 further comprising a shutter between said third focusing lens and set fourth focusing lens.

45. The laser apparatus of claim 41 wherein said first cylindrical lens is a negative lens, and said second cylindrical lens is a positive lens.

46. Myocardial revascularization apparatus comprising the laser apparatus of claim 39 and a laser delivery system for delivering laser pulses to a patient's heart to provide openings in the patient's heart.

47. The apparatus of claim 46 wherein said laser delivery system includes a hand piece for delivering pulses to the outside of a patient's heart.

48. A slab laser comprising
a pair of elongated spaced apart electrodes having opposed planar surfaces defining a discharge region between said opposed planar surfaces, said electrodes having aligned side surfaces extending along a longitudinal axis thereof, and
a plurality of nonconductive spacers each connecting one said electrode to the other said electrode at said side surfaces and being spaced from adjacent spacers along said side surfaces,
said spacers having relieved portions facing said discharge region between first and second planes passing through said planar surfaces, said relieved portions including at least one projecting portion extending toward said discharge region and having an apex closer to said discharge region than adjacent portions of said relieved portions.

49. The laser of claim 48 wherein there is a third plane including said side surfaces and wherein said apex is on the side of said third plane outside of said discharge region and is spaced from said third plane.

50. The laser of claim 48 wherein each said spacer has at least two said projecting portions extending toward said discharge region and having an apex region.

51. The laser of claim 48 wherein said apex converges to a line.

52. Myocardial revascularization apparatus comprising the laser of claim 48 and a laser delivery system for delivering laser pulses to a patient's heart to provide openings in the patient's heart.

53. The apparatus of claim 52 wherein said laser delivery system includes a hand piece for delivering pulses to the outside of a patient's heart.

54. A slab laser comprising
a pair of elongated spaced apart electrodes having opposed planar surfaces defining a discharge region between said opposed planar surfaces, said electrodes having aligned side surfaces extending along a longitudinal axis thereof,
a plurality of nonconductive spacers each connecting one said electrode to the other said electrode at said side surfaces and being spaced from adjacent spacers along said side surfaces,
a plurality of barbed inserts in said side surfaces and spaced from adjacent barbed inserts along said surfaces, and
a plurality of inductors that each have one end inserted into a said barbed insert on one electrode and another end inserted into a said barbed insert on another electrode.

55. Myocardial revascularization apparatus comprising the laser of claim 54 and a laser delivery system for delivering laser pulses to a patient's heart to provide openings in the patient's heart.

56. The apparatus of claim 55 wherein said laser delivery system includes a hand piece for delivering pulses to the outside of a patient's heart.

57. A slab laser comprising
an elongated housing having an internal ledge structure along at least one side,
a pair of elongated spaced apart electrodes having opposed planar surfaces defining a discharge region between said opposed planar surfaces, and
a plurality of resilient contact elements extending along a bottom of a said electrode and supported on said internal ledge structure to provide good electrical connection between said electrode and said housing.

58. The slab laser of claim 57 wherein said electrodes have aligned side surfaces extending along a longitudinal axis of said housing, and a further comprising a plurality of nonconductive spacers each connecting one said electrode to the other said electrodes at said side surfaces and being spaced from adjacent spacers along said side surfaces.

59. Myocardial revascularization apparatus comprising the laser of claim 57 and a laser delivery system for delivering laser pulses to a patient's heart to provide openings in the patient's heart.

60. The apparatus of claim 59 wherein said laser delivery system includes a hand piece for delivering pulses to the outside of a patient's heart.

61. Laser apparatus comprising
a housing defining an elongated laser chamber therein extending along a longitudinal axis,
a frame having support portions supporting said housing, and
isolation mounts between said support portions and said housing.

62. The apparatus of claim 61 further comprising wheels attached to said frame.

63. The apparatus of claim 61 wherein said bumpers include a vertically oriented bumper and a horizontally oriented bumper.

64. The apparatus of claim 61 wherein said bumpers include two vertically oriented bumpers and two horizontally oriented bumpers.

65. The apparatus of claim 61 wherein said housing is the housing of a slab laser.

66. The apparatus of claim 61 further comprising a laser delivery system for delivering laser pulses to a patient's heart.

67. The apparatus of claim 66 wherein said laser delivery system includes a hand piece for delivering pulses to the outside of a patient's heart to provide openings in the patient's heart.

68. The apparatus of claim 67 wherein said laser delivery system includes an articulated arm that has one end that is supported by said housing, and another end that carries said hand piece.

69. Myocardial revascularization apparatus comprising
a laser,
a laser delivery system for delivering laser pulses from a hand piece to a patient's heart,
an air source including a compressor and an air filter, and
a tube connected to said laser delivery system to deliver purge air from said source to said hand piece to clear smoke from the inside of said hand piece.

70. The apparatus of claim 69 wherein said laser delivery system includes an articulated arm that has one end that is supported by said laser, and another end that carries said hand piece, and wherein said arm carries said tube.

71. Laser apparatus comprising
a housing defining an elongated laser chamber therein extending along a longitudinal axis,
a support supporting said housing with said axis aligned substantially with a vertical axis,
said housing having a laser output window at the lower end of said housing, and
said housing having an upwardly extending lip around said window to limit collection of particles on said window.
